(19) United States
(12) Patent Application Publication
(10) Pub. No.: US 2022/0280113 A1
(43) Pub. Date: Sep. 8, 2022

(54) RESPIRATORY SENSOR PATCH

(71) Applicant: Mission Support and Test Services, LLC, Las Vegas, NV (US)

(72) Inventors: Scott B. Sonnon, North Las Vegas, NV (US); Erik Stassinos, North Las Vegas, NV (US)

(21) Appl. No.: 17/688,535

(22) Filed: Mar. 7, 2022

Related U.S. Application Data
(60) Provisional application No. 63/157,504, filed on Mar. 5, 2021.

(51) Int. Cl. A61B 5/00; A61B 5/087

(52) U.S. Cl. CPC A61B 5/6833; A61B 5/0873; A61B 2562/223

(57) ABSTRACT

A respiration monitor configured as a patching having adhesive on one side to allow adhesive attachment of the patch to a person. The patch includes a transmitter which generates a reference signal that is provided to a signal path. The signal path is configured to receive and conduct a transmitted reference signal such that the signal path has a length that increases when the person inhales and contracts when the person exhales. A receiver is configured to receive a received reference signal from the signal path. A controller is configured to control generation and transmission of the reference signal and processing the transmitted reference signal and the received reference signal to identify changes in the received reference signal due to transmission through the signal path as the signal path expands and contracts. This is done to generate respiration data that reflects respiration characteristics of the person.

### 1. FIELD OF THE INVENTION

The innovation relates to respiratory sensor, and in particular to a method and apparatus for precise respiration sensing using an adhesive patch with movement sensors.

### 2. RELATED ART

Numerous situations exist where monitoring breathing can provide helpful insight into a person's health and exertion level. Numerous devices have been proposed to monitor respiration. However, these devices suffer from many drawbacks.

One such drawback is that prior art respiration devices are bulky, heavy, and difficult to use during activity. Example of such devices are systems which are strapped to the user, such as around the chest, and detect movement of the device as the user breaths. Other prior art devices include systems which the user breaths through while it monitors one or more aspects of respiration. While these devices do monitor respiration, they suffer from being bulky, heavy, and are not suitable for use when the user is active, or for extended periods of time.

In addition, prior art solutions do not monitor sufficient respiration aspects to provide a full account of the user's health and respiration related physical aspects. For example, prior art methods monitor respirator rate, but this factor does not provide a full understanding of respiration in relation to a user's physical state. Therefore, there is a need in the art for a respiration method and apparatus which overcomes the drawbacks of the prior art.

### SUMMARY

To overcome the drawbacks in the prior art and provide additional benefits, a respiratory sensor patch is disclosed which collects data in real-time. Patch fibers act as a strain gauge to detect excursion occurring through movement of breathing. Sensor application is superior to navel, midline to sagittal plane; and on the ribcage, over the left-side costal cartilage.

The collected data may include respiratory rate, volume (tidal and minute ventilation), ratio of abdominal vs thoracic excursion, breath cycle ratio (duration of inspiration, post-inspiratory hold, expiration and post-expiratory hold), respiratory rhythm (variability) and cardio-respiratory synchronization (variability of heart rate, breath rate and breath volume). The adhesive, or otherwise attachable patch form factor, detects changes in raw data signals and derived parameters due to the excursion of piezo-electric strain gauge fiber-optics in the fabric of the patch, or through use of any other sensor type. The patch may be applied lateral to the lower ribs on the external intercostal muscles, and to the abdomen (belly area), below the navel. The data may be portrayed in a user device graphical interface for in-situ mitigation of stress response. The data may be transferred via wireless communication to a laptop or other processing device for real-time remote monitoring and transferred to cloud storage or computing environment for synchronous data management and cross-queuing with other biosensor data analysis.

To overcome the drawbacks of the prior art and provide additional benefits, a respiration monitor is disclosed. In one embodiment, the respiration monitors comprise a patching having a first side and a second side. The first side has an adhesive thereon to allow adhesive attachment of the patch to a person. A transmitter is part of the respiration monitor, and is configured to generate a reference signal that is provided to a signal path. The signal path is configured to receive and conduct a transmitted reference signal such that the signal path has a length that increases when the person inhales and contracts when the person exhales. A receiver is configured to receive a received reference signal from the signal path. A controller is configured to control generation and transmission of the reference signal to the signal path. The controller is also configured to process the transmitted reference signal and the received reference signal to identify changes in the received reference signal due to transmission through the signal path as the signal path expands and contracts. The controller also generates respiration data that reflects respiration characteristics of the person. A power source is configured provide power to the transmitter, receiver, and controller.

In one embodiment, the controller is a processor and the respiration monitor further comprises a memory storing non-transitory machine executable code, such that the machine executable code is configured to be executed by the processor to process the transmitted reference signal and the received reference signal. In one embodiment, the signal path comprises a fiber optic cable. It is contemplated that the transmitter may comprise an optic transmitter. In one configuration, the respiration monitor further comprises a wireless transmitter configured to wirelessly transmit respiration data from the respiration sensor to a separate receiver. The second side may include a protective layer configured to cover the transmitter, signal path, receiver, controller and power source.

Also disclosed is a respiration monitor comprising a power source and at least one strain sensor configured to generate an output signal indicating strain experience by the at least one strain sensor. A controller configured to execute machine executable code which is read from a memory. The memory is configured to store the machine executable code which is readable by the controller. The machine executable code is configured to process the output signal from the at least one strain sensor to generate strain data indicating expansion and contraction of the at least one strain sensor, as well as transmit or facilitate reading of the strain data from the respiration monitor for processing by a computing device. These elements are part of a patch that has a first side and a second side, the first side configured to adhesively attach to a person, and the second side configured to overlay and protect the at least one strain sensor, controller, and memory which are located between the first side and the second side.

The one or more strain sensors may be selected from the group of quarter bridge strain gage, strain gauge rosettes, piezo resistor, liquid metal, Raman shift and triboelectric, capacitive, resistive, or any other type of strain gauge, or any other type strain gauge or sensor. The power source may comprise a battery. It is also contemplated that the respiration monitor may further comprise a transceiver configured to wirelessly communicate the strain data to an external computing device. In one configuration the respiration monitor is configured to detect abdominal band movement and thoracic band movement during respiration by the person wearing the respiration monitor.

Also disclosed is a method for monitoring respiration. Provided as part of the method, is a respiration monitor comprising expansion and contraction sensors which are part of an adhesive patch. The patch is adhesively attached to the respiration monitor to an abdomen, thorax, or both of a person and an activation signal is sent to the respiration monitor. The respiration monitor then generates a reference signal which is provided to a strain sensor and an output signal is output from the strain sensor. The method of operation processes the output signal to detect expansion data and contraction data of the strain sensor due to respiration of the person. The expansion data and contraction data is transmitted to a computing device that is separate from the respiration monitor wherein the data is processed to identify abdominal band movement, thoracic band movement, or both which represent respiration of the user.

In one embodiment this method further comprises processing the abdominal band movement, thoracic band movement, or both to generate one or more of the following: respiration rates, ratio of hold time exhale, pause time inhale, inhale time, and exhale time, percent of Ribcage Excursion, Tidal Volume, Minute Volume, Inhale Airflow Velocity Exhale Airflow Velocity, Respiratory Rhythm, and Respiratory Synchrony. It is contemplated that the strain sensor may comprise a light conductor which changes transfer function based on excursion of the strain sensor. In one configuration the strain sensor comprises a strain gauge which changes resistance based on excursion of the strain gauge.

The processing may be performed by a processor executing machine executable code stored in non-transitory form on a memory. In addition, the expansion data and contraction data, or data derived therefrom, may be shown graphically to represent respiration of the person. The step of transmitting may comprise establishing an electrical connection between the respiration monitor and the computing device.

Other systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims.

### BRIEF DESCRIPTION OF THE DRAWINGS

The components in the figures are not necessarily to scale, emphasis instead being placed upon illustrating the principles of the invention. In the figures, like reference numerals designate corresponding parts throughout the different views.

FIG. 1 illustrates an overview of an exemplary respiration device and method of use.

FIG. 2 illustrates an example embodiment of a patch applied to the skin.

FIG. 3A illustrates an exemplary sensor type that may be used with the patch described herein.

FIG. 3B illustrates the same sensor type shown in FIG. 3A but repeatedly stretched to a length D1 and then relaxed to a distance D2.

FIG. 3C illustrates an exemplary plot of signal power loss, after passing through the light conductor for various amounts of elongations.

FIG. 4 illustrates a block diagram of the light conductor and associated sensor system of an example embodiment of a respiratory sensor patch.

FIG. 5 illustrates an alternative embodiment of the respirator sensing patch.

FIG. 6 illustrates an exemplary plot of signals derived from respiration monitoring with associated respiration aspects calculations.

FIG. 7 illustrates an exemplary plot of the dynamic ratio of thoracic versus abdominal movement.

### DETAILED DESCRIPTION

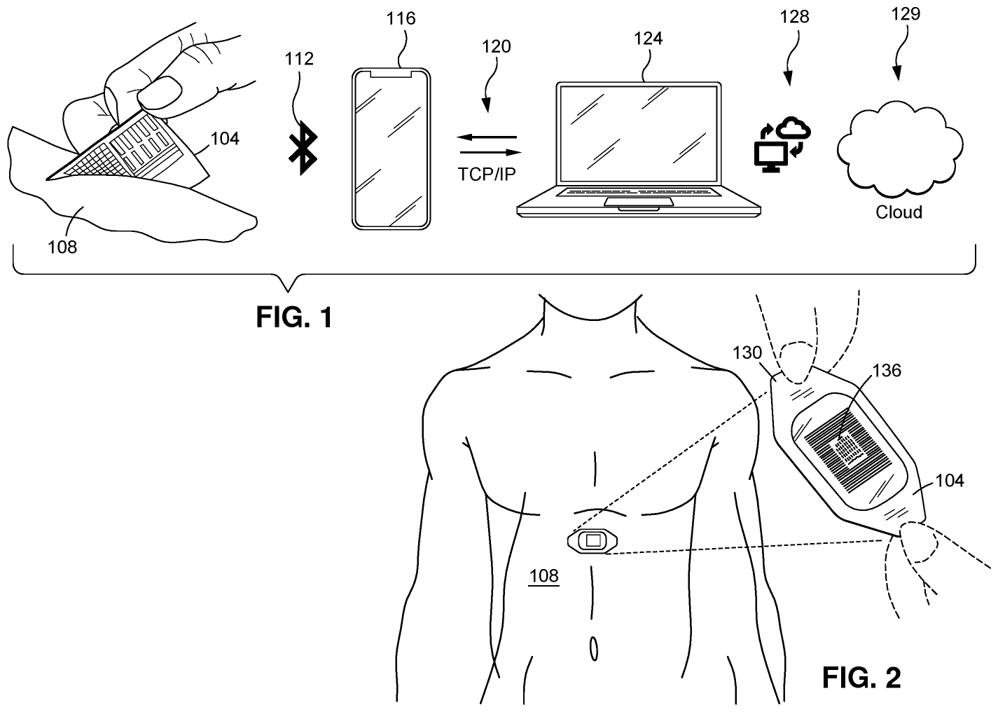

To overcome the drawbacks of the prior art and provide additional benefits, disclosed is a body attachable respiration system and method. FIG. 1 illustrates an overview of an exemplary respiration device and method of use. As shown, a patch 104 is attached on skin 108 of a user. The patch 104 has movement sensor(s) attached or imbedded therein to monitor movement of the patch, or one area of the patch, in relation to another area of the patch. The patch may have wireless communication electronics to wirelessly communicate 112 to a nearby receiver 116. In one embodiment, the communication occurs using the Bluetooth communication protocol. The receiver 116 may be any type electronic device such as a smartphone, wearable electronics, or purpose built communication device. The receiver 116 may perform processing on the data from the patch 104 and may optionally provide feedback to the user.

The receiver 116 may be configured to upload the data to a remote terminal 124, such as a laptop, computer, vehicle, aircraft, drone, server or any other computing device. The data may be further processed at the remote terminal 124 for display on a monitor of the remote terminal 124 to present the data in graphics for best utilization. It is also contemplated that the data may be uploaded to a cloud server or database 128 from the remote terminal 124 or directly from the receiver 116. The transmission to the cloud 129 may occur over any type of connection such as the Internet, private network, cellular network, or satellite network using any known or future developed communication protocol.

Numerous example environments of use are possible including but not limited to the medical field, military applications, sports training, and aviation (pilot monitoring—hypoxic situations—cardio-respiratory behavior), or any situation, where air quality or availability is at question (climbing, industry, chemical, drivers, transportation).

FIG. 2 illustrates an example embodiment of a patch 104 applied to the skin 108. The patch may have an outer ring or band which is used for application and removal from the user. The inner area of the patch 104 may include adhesive or any other means to attach and secure the patch to the user 108. In the inner area 136 of the patch are one or more electronic elements and one or more sensors configured to precisely monitor movement, such that the movement can be translated into respiration data.

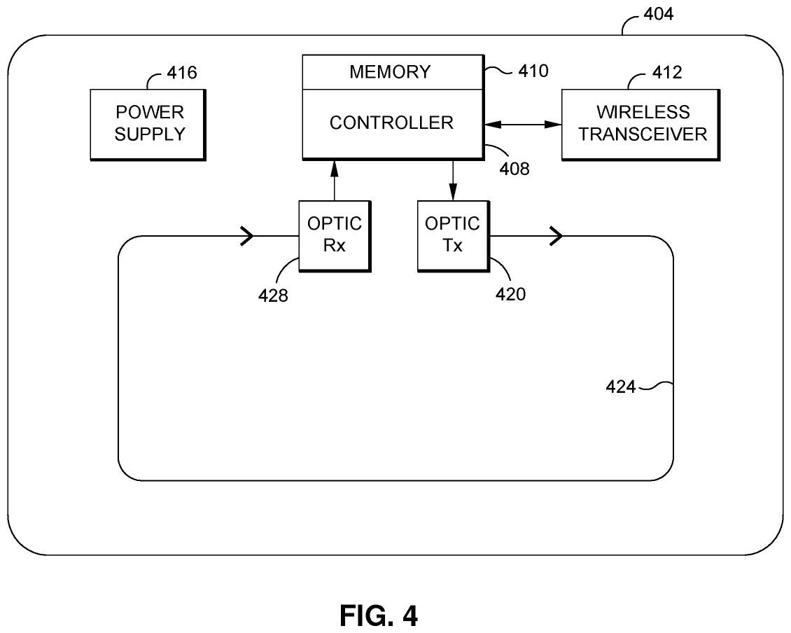

The patch may be made from any durable material configured to contain and protect patch sensor elements. The patch may be any size such as, but not limited to, one inch by one inch up to larger sizes as may adhesively attach to the user. Adhesive attachment has the benefit of securing the entire patch to the user such that expansion of the user's chest area causes the patch to likewise expand or be strained. In one embodiment, the patch attaches to the user on the left-hand side of the lower ribs, between two ribs and lateral to the heart. The patch may also be placed on the stomach area, beneath the navel, under the belt line. It is contemplated that other area(s) may be used for patch placement which are suitable for monitoring respiration and other body functions. FIG. 4 describes the patch in greater detail.

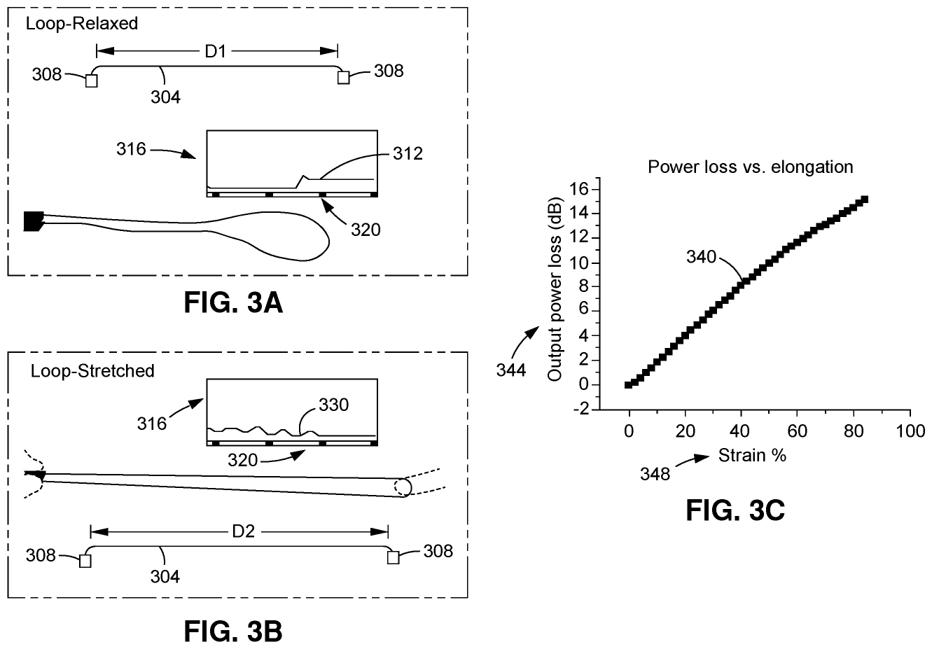

FIG. 3A illustrates an exemplary sensor type that may be used with the patch described herein. This is but one possible type sensor and any other sensor types may be used with the patch described herein. In this embodiment, a light conductive sensor includes a light conductor 304 with a connector 308 on each end. The light conductor may comprise a fiber optic cable configured to behave as described herein. When in a rest or unstretched length D1, a signal transmitted through the light conductor 304 is represented by the plot 312 with the vertical axis 316 representing signal magnitude and the horizontal axis representing time. As can be seen, when in a relaxed position the light conductor 304 has a length D1 and the output power is generally constant. The output power is defined as the output power of the optic signal passing through the light conductor 304.

In contrast, FIG. 3B illustrates the same sensor type shown in FIG. 3A but repeatedly stretched to a length D1 and then relaxed to a distance D2. As can be seen in plot 330, the output power varies in direct relation to the amount of elongation of the light conductor 304.

FIG. 3C illustrates an exemplary plot of signal power loss, after passing through the light conductor 304, for various amounts of elongations. In FIG. 3C the vertical axis 344 represents output power loss, while the horizontal axis represents strain (elongation) of the light conductor 304. As shown in plot 340, as strain increases, the output power loss also increases, and the relationship is generally linear. Thus, the output power of the light signal passing through the light conductor 304 is reduced as the light conductor is stretched. Using this relationship, the light conductor can be used as a high precision strain sensor. When combined with a patch as described in FIG. 2, the respiratory patterns of a user can be monitored with great precision providing data previously not available in the prior art with a small, wearable, form factor.

FIG. 4 illustrates a block diagram of the light conductor and associated sensor system of an example embodiment of a respiratory sensor patch. This is but one possible embodiment and other configurations are contemplated. In this embodiment, a controller 408 is provided to oversee operation of the patch 404. The controller 408 may comprise a processor, control logic, ASIC, micro-controller, or any other similar device. The controller 408 may executed machine readable code stored on a memory that is part of the controller or a separate element.

The controller 408 connects to a wireless transceiver 412 configured to wirelessly communicate data from the sensor patch 404 to an external receiver (not shown). The wireless transceiver 412 may comprise any type system as would be understood in the art, such as but not limited to, Bluetooth communication standards and any other protocol or systems. The communications system may be configured with bi-directional communication capability, such as to be used for the onboard data processor to recognize a host wireless device and transmit. It is contemplated that the patch may be configured to communicate with any device, such as an Android OS device, Microsoft OS device, Unix, Linux, iOS, or any other type device of operating system.

It is also contemplated that the data may be stored in a memory 410 that is accessible by the controller 408. In such an embodiment, the data may be stored and accessed at a later time, such as through direct electrical connection or an RFID interface. The wireless transceiver 412 may be eliminated in embodiments which store the respiration data to reduce codes, size, power consumption and complexity.

The controller 408 is configured to present a reference optic signal to an optic transmitter 420. The optic transmitter 420 may comprise a LED or laser or any other type of optic signal generator. The reference optic signal is transmitted into the light conductor 424, which may be arranged in any manner or path to conduct the optic signal to an optic signal receiver 428. The light conductor may be formed from polyurethane and silicon fibers configured to stretch, when adhesively attached to the user, during inspiration and the excursion creates an amplitude signal representative of breathing rate and volume. The release of the stretch occurs during expiration, and that decreased excursion creates a signal that is representative of breathing rate and volume. When those two signals are analyzed by software, the excursion/stretch, the sloping off of the excursion, the decrease in the excursion, and the sloping off of the decreased excursion, create respiration data that represent the entire breath cycle: inspiration, post-inspiratory retention, expiration, and post-expiratory suspension.

As the signal passes through the light conductor 424, respiration is also occurring, and the expansion and contraction of the chest causes the light conductor to repeatedly stretch (strained) and return to an un-stretched (unstrained) state. As discussed herein, this changes the power of the reference signal received by the optic receiver 428.

The optic receiver 428 converts the optic signal, with a photodetector, to an analog electrical signal and presents the electrical signal to the controller 408. An analog to digital converter (ADC) may be part of the controller 408 or the optic receiver 428 to convert the analog output from the photodetector (such as a photodiode) to a digital signal suitable for processing by the controller. The controller 408 can compare the transmitted reference signal to the received reference signal to determine the changes in power level and extrapolate respiration data. Thus, the patch is a part of an electro-optical system converting light as it passes through optical fibers to a voltage signal read by an onboard controller. Variance in the voltage, over time, reflects user respiration patterns.

A power source 416 provides power to all the elements of the patch 404 that require power. Any type power source may be utilized including but not limited to battery, solar with extension cable and solar cell, chemical based electrical generation, body heat electrical generation, electrical generating clothing, or motion generated power. It is contemplated that additional elements may be provided which are not shown but which would be understood by one of ordinary skill in the art to enable operation. Such elements include but are not limited to memory, additional conductor, buffers, clock generators, capacitors, resistors, inductors, or any other device.

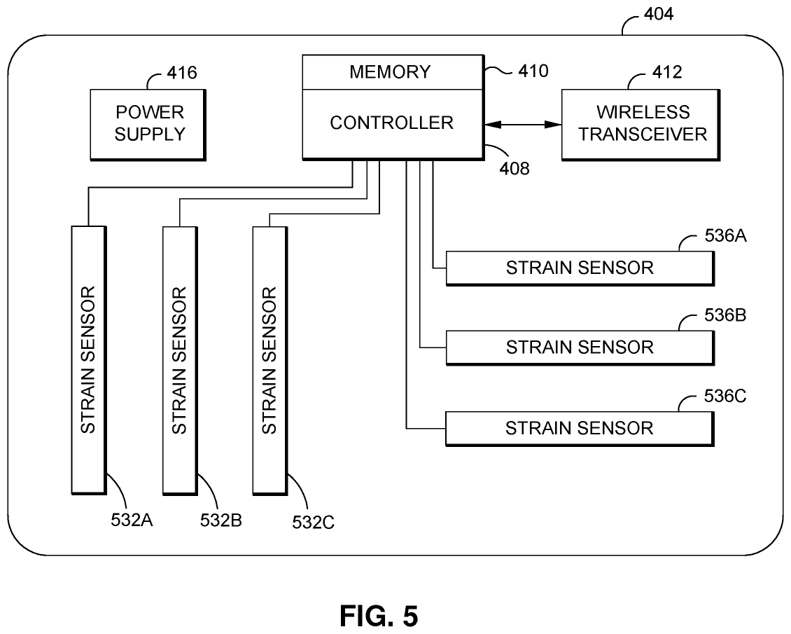

FIG. 5 illustrates an alternative embodiment of the respirator sensing patch. As compared to FIG. 4, similar elements are identified with identical reference numbers. Although the patch is described in connection with a light conductive sensor, it is contemplated that other types of sensors may be used to detect the small motion of user respiration. In this example embodiment, the light conductor strain sensor is replaced with one or more strain sensors 532, 536 which senses stretching in the horizontal direction and the vertical direction. Multiple strain sensors are shown to have greater accuracy in the sensor and as back up in case of failure or faulty data. The strain sensors may be arranged in any physical layout with the shown pattern being exemplary only. Any type strain gauge may be used, including but not limited to, a quarter bridge strain gauge, strain gauge rosettes, piezo resistor, liquid metal, Raman shift and triboelectric, capacitive, resistive, or any other type of strain gauge.

In operation, the patch is verified to be operational and the portion of the user's body to which the patch will attach is cleaned to provide an optimal surface for adhesion. Other methods of sensor attachment are contemplated and possible. The one or more patches are then placed at a location on the user selected to sense the various stages of respiration. The patch detects the respiration and transmits the raw respiration data to a receiver.

As stated above, the raw signals will be representative of the four aspects of the breath cycle. This is also known as the breath ratio, portrayed as a value in seconds per aspect, e.g., 4062 breath cycle defined as 4 second inhale, no post-inspiratory retention, a 6 second exhale, and a 2 second post-expiratory suspension. This is an important theoretical respiratory parameter in identifying breathing phenotypes for differing cognitive, emotional, exertional and speech behaviors.

Derived from this breath cycle (4062 breath cycle) is also a respiratory rate (f: frequency of stretch cycle representative of breath cycle extrapolated in breaths/min) and respiratory volume (TV: amplitude of stretch extrapolated to milliliters). Finally, when TV is extrapolated over a projected minute, it serves as another parameter, minute ventilation (MV). The TV and MV are the conventional/known breathing parameters used in current commercial wearable monitors, but these variables do not provide a full assessment of respiration.

Derived from the breath cycle will also be inspiratory airflow velocity which is defined as the time in seconds of the stretch of the material on inspiration (Ti) over the amount of the stretch of the material on inspiration (TV) equals the airflow velocity (Vi). Therefore, Vi=TV/Ti. Additionally, the corollary for expiratory airflow velocity is defined as Ve=TV/Te.

In some embodiments, two patches are attached to the user. The two patches may be placed at various different locations, such as one on the abdomen and one on the thorax, a percentage difference between the excursion of the two patches will represent the level of distress/arousal of the individual's breathing. The degree to which accessory breathing muscles are recruited, the degree to which the total excursion of the two bandages will be generated from the thoracic (compared to the abdominal) patch represent the level of distress/arousal of the individual's breathing. That percentage of ribcage excursion on inspiration (% RCi) is a critical parameter of respiration.

Lastly, variance (VAR) in the signal behaviors across minutes and hours (and longer) represents two critical theoretical parameters for respiration. For example, rhythmicity is determined by analyzing the breathing frequency (f) and breathing volume (MV) over time, variance or invariance can be detected in the pattern of the signals, creating a rhythm to the waveform portrayed on the graphical user interface. When the pattern of f and MV are invariant, certain health conclusions can be made; and when the pattern is highly varied, distress/arousal can be presumed. This information and the patterns can be represented in arrhythmicity in the waveform in the graphical user interface. Also disclosed is synchrony, which when analyzing the relationship of frequency and volume changes in breathing, occurs when the signal comparison displays invariance, then conclusions can be drawn about the health of the wearer/user. When the comparison of the two signals is highly variant, then distress/arousal can be presumed.

Once calculated, the data may be aggregated and converted to a time-based visualization scheme where some error analysis is done to disqualify outlier data. Data is displayed with visualization tools to the user such as on a computer display to provide real time, or after event data regarding respiration and/or other performance factors. The displayed information can be a continuous data stream of aggregate data from each patch within the patch system. Of each sinusoidal data set, statistics will include, but are not limited to, patch peak value, patch minimum values, variance in periodicity, variance in amplitude, peak width, through width, and second and third derivative statistics of the sensor data correlated within a region of interest on time series. The data may relate to operational concepts stated above. A proprietary graphical user interface will display the information for the user with one or more warning or alert levels contemplated to provide notice when the respiration data is outside of a predetermined range. The information will have potential to be stored for long term analysis or can be utilized as a trigger source for non-visual and/or visual user feedback.

It is also contemplated that additional data may be collected beyond respiration data with the disclosed patch or with ancillary monitoring systems. Such data includes but is not limited to, heart rate, heart intensity, electrocardiograms data, nerve activity, perspiration, temperature, pressure, digestive function, blood oxygen levels, end-tidal CO₂, (sub-cutaneous), carbon dioxide, venous or arterial blood gas levels, or any other parameter. It is also contemplated that the processing of the data may include synchronization between signals such as, but not limited to, cross-queuing or cross comparing signals—get heart and multiple respiration signals and monitor/analyze how those signals synchronize and how those signals vary over time in relation to each other. It is contemplated that any signals or data disclosed herein may be compared and contrasted to generate additional data and relationships for performance and health. The prior art does not compare and contrast these signals and no correlation of or between data exists in the prior art.

It is also contemplated that the sensor disclosed herein may be used to collect base line data regarding a user's respiration and/or compare to later collected respiration parameters for analyzing based on comparison data across all users.

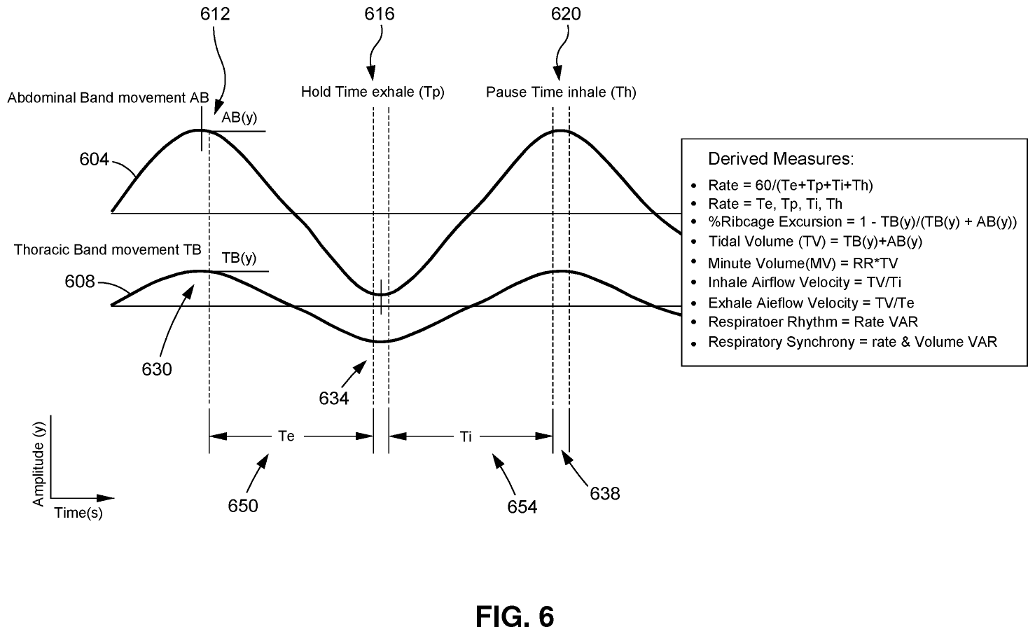

FIG. 6 illustrates an exemplary plot of signals derived from respiration monitoring with associated respiration aspects calculations. As shown in FIG. 6, an abdominal band movement plot 604 is shown over time during respiration as monitored by the monitor described herein. As shown, an abdominal band movement peak 612 is shown which is maximum expansion or extension of the chest or abdomen. An exhale hold time 616 is shown as the time between exhale and inhale. Similarly, an inhale pause time 620 is shown as the time between inhale and exhale. All of this data may be collected with the respiration monitor described herein.

Also shown in FIG. 6 is a thoracic band movement plot 608. This plot 608 includes a peak thoracic band movement 630. For the thoracic band movement, an exhale hold time 635 is shown as the time between exhale and inhale. Similarly, an inhale pause time 638 is shown as the time between inhale and exhale. All of this data may be collected with the respiration monitor described herein. From these plots, numerous additional respiration details may be calculated. Using the information from the plots shown in FIG. 6 over time, the following important respiration parameters may be calculated: respiration rates, ratio of hold time exhale, pause time inhale, inhale time, and exhale time, percent of ribcage excursion, tidal volume, minute volume, inhale airflow velocity, exhale airflow velocity, respiratory rhythm, and respiratory synchrony.

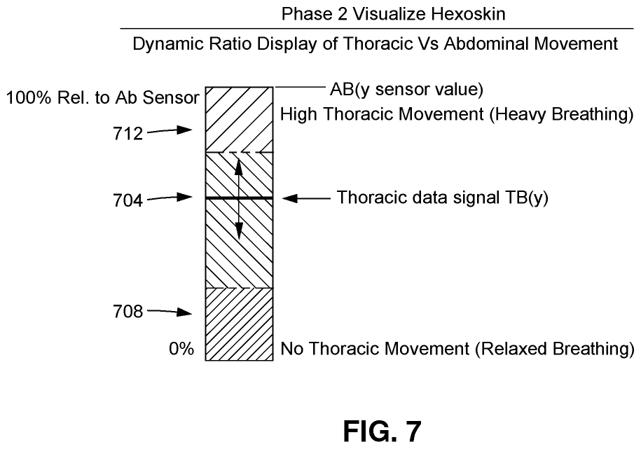

FIG. 7 illustrates an exemplary plot of the dynamic ratio of thoracic verses abdominal movement. As shown, high thoracic movement is associated with heavy breathing, while no or low thoracic movement is associated with relaxed breathing. The thoracic data signal represents the chest or abdomen movement and is thus correlated to physical breathing level based on the sensor data. In FIG. 7 the thoracic data signal 704 will change as the user breaths in and out. Thus, the thoracic data signal 704 will move up or down, as indicated by the arrows, as respiration occurs and as monitored by the monitoring patch described herein. During periods of shallow or light respiration, the thoracic data signal 704 will not rise out of the lower area 708. During periods of deep or heavy respiration, the thoracic data signal 704 will not rise out of the lower area 708 and into the upper area 712.

While various embodiments of the invention have been described, it will be apparent to those of ordinary skill in the art that many more embodiments and implementations are possible that are within the scope of this invention. In addition, the various features, elements, and embodiments described herein may be claimed or combined in any combination or arrangement.

What is claimed is:

1. A respiration monitor comprising:
a patching having a first side and a second side, the first side having an adhesive thereon to allow adhesive attachment of the patch to a person;
a transmitter configured to generate a reference signal that is provided to a signal path;
a signal path configured to receive and conduct a transmitted reference signal, the signal path having a length that increases when the person inhales and contracts when the person exhales;
a receiver configured to receive a received reference signal from the signal path;
a controller configured to:
control generation and transmission of the reference signal into the signal path;
processing the transmitted reference signal and the received reference signal to identify changes in the received reference signal due to transmission through the signal path as the signal path expands and contracts;
generating respiration data that reflects respiration characteristics of the person; and
a power source configured to provide power to the transmitter, receiver, and controller.

2. The respiration monitor of claim 1 wherein the controller is a processor and the respiration monitor further comprises a memory storing non-transitory machine executable code, the machine executable code configured to be executed by the processor to process the transmitted reference signal and the received reference signal.

3. The respiration monitor of claim 1 wherein the signal path comprises a fiber optic cable.

4. The respiration monitor of claim 1 wherein the transmitter comprises an optic transmitter.

5. The respiration monitor of claim 1 further comprising a wireless transmitter configured to wirelessly transmit respiration data from the respiration sensor to a separate receiver.

6. The respiration monitor of claim 1 wherein the second side includes a protective layer configured to cover the transmitter, signal path, receiver, controller and power source.

7. A respiration monitor comprising:
a power source;
at least one strain sensor configured to generate an output signal indicating strain experience by the at least one strain sensor;
a controller configured to execute machine executable code;
a memory configured to store the machine executable code which is readable by the controller, the machine executable code configured to:
process the output signal from the at least one strain sensor to generate strain data indicating expansion and contraction of the at least one strain sensor;
transmit or facilitate reading of the strain data from the respiration monitor for processing by a computing device; and
a patching having a first side and a second side, the first side configured to adhesively attach to a person, and the second side configured to overlay and protect the at least one strain sensor, controller, and memory which are located between the first side and the second side.

8. The respiration monitor of claim 7 wherein the one or more strain sensors is selected from the group of quarter bridge strain gage, strain gauge rosettes, piezo resistor, liquid metal, Raman shift and triboelectric, capacitive, resistive, or any other type of strain gauge.

9. The respiration monitor of claim 7 wherein the power source comprises a battery.

10. The respiration monitor of claim 7 further comprising a transceiver configured to wireless communicate the strain data to an external computing device.

11. The respiration monitor of claim 7 wherein the respiration monitor is configured to detect abdominal band movement and thoracic bank movement during respiration by a person wearing the respiration monitor.

12. A method for monitoring respiration comprising:
providing a respiration monitor comprising expansion and contraction sensors established as part of an adhesive patch;
adhesively attaching the respiration monitor to an abdomen, thorax, or both, of a person;
sending an activation signal to the respiration monitor;
generating a reference signal;
providing the reference signal to a strain sensor;
receiving an output signal from the strain sensor;
processing the output signal to detect expansion data and contraction data of the strain sensor due to respiration of the person;
transmitting the expansion data and contraction data to a computing device that is separate from the respiration monitor;
processing the expansion data and contraction data to identify abdominal band movement, thoracic band movement, or both, which represent respiration of the user.

13. The method of claim 12 further comprising processing the abdominal band movement, thoracic band movement, or both, to generate one or more of the following: respiration rates, ratio of hold time exhale, pause time inhale, inhale time, and exhale time, percent of ribcage excursion, tidal volume, minute volume, inhale airflow velocity, exhale airflow velocity, respiratory rhythm, and respiratory synchrony.

14. The method of claim 12 wherein the strain sensor comprises a light conductor which changes transfer function based on excursion of the strain sensor.

15. The method of claim 12 wherein the strain sensor comprises a strain gauge which changes resistance based on excursion of the strain gauge.

16. The method of claim 12 wherein the processing is performed by a processor executing machine executable code stored in non-transitory form on a memory.

17. The method of claim 12 further comprising graphically plotting the expansion data and contraction data or data, derived therefrom, to represent respiration of the person.

18. The method of claim 12 wherein the transmitting comprising establishing an electrical connection between the respiration monitor and the computing device.
